(19) United States
(12) Patent Application Publication
(10) Pub. No.: US 2006/0040873 A1
(43) Pub. Date: Feb. 23, 2006

(54) Composition for use in prophylaxis and or treatment

(75) Inventors: Neil Roland McGregor, Eleebana (AU); Henry Lawrence Butt, Backburn (AU); Richard Hugh Dunstan, Ellermore Vale (AU); Timothy Roberts, Newcastle (AU)

Correspondence Address: SUGHRUE MION, PLLC, 2100 PENNSYLVANIA AVENUE, N.W., SUITE 800, WASHINGTON, DC, 20037, US

(73) Assignee: BIOREVIVE PTY., LTD., Victoria (AU)

(21) Appl. No.: 10/483,499

(22) PCT Filed: Jul. 5, 2002
(86) PCT No.: PCT/AU02/00894

§ 371 Date: Oct. 26, 2004

(30) Foreign Application Priority Data
Jul. 10, 2001 (AU) PR 6262

(51) Int. Cl. A61K 31/70; A61K 31/7008; A61K 31/198; A61K 31/721

(52) U.S. Cl. 514/23; 514/59; 514/561; 514/62; 514/58

(57) ABSTRACT

The present invention relates generally to a composition for use in the prophylaxis and/or treatment of pain. More particularly, the present invention relates to a composition for use in the prophylaxis and/or treatment of chronic neuromuscular pain. The composition of the present invention is particularly useful in the prophylaxis and/or treatment of chronic neuromuscular pain such as, for example, fibromylagia, myofascial pain, repetitive strain injury or overuse syndromes, and cytokine-mediated cancer and chemotherapy associated pain.

### FIELD OF THE INVENTION

The present invention relates generally to a composition for use in the prophylaxis and/or treatment of pain. More particularly, the present invention relates to a composition for use in the prophylaxis and/or treatment of chronic neuromuscular pain. The composition of the present invention is particularly useful in the prophylaxis and/or treatment of chronic neuromuscular pain such as, for example, fibromylagia, myofascial pain, repetitive strain injury or overuse syndromes, and cytokine-mediated cancer and chemotherapy associated pain.

### BACKGROUND OF THE INVENTION

Bibliographic details of the publications referred to by author in this specification are collected at the end of the description.

Reference to any prior art, in this specification is not, and should not be taken as an acknowledgment or any form of suggestion that this prior art is common general knowledge or forms a part of the common general knowledge in Australia or any other country.

Chronic neuromuscular pain is a very common clinical and therapeutic problem (de Girolamo, 1991) which affects up to 70% of the population, with severe forms, such as fibromyalgia, occurring between 5-10%. Myofascial pain syndrome occurs in approximately 45-50% of the community with pain of sufficient intensity being noted in 15-20% of the population. The degree of pain or discomfort appears to dictate patient reporting and treatment presentation. These syndromes occur with low prevalence until post puberty, after which they increase in prevalence and severity in the 30 to 50 year age group with a small decline following 50 years of age. There is a female:male ratio between 3.5:1 and 4:1 with the percentage of females increasing with increasing severity of the pain. In patients with fibromyalia, a broad range of symptoms are also reported including: sleep disturbance, fatigue, depression, gastrointestinal disturbance, sinus and thyroid problems, concentration problems, swollen glands, tachycardia and weakness as well as joint hypermobility indicating a whole body metabolic problem (Waylonis & Heck, 1992). Between 60-65% of these chronic neuromuscular pain patients have a slow or gradual onset of symptoms with a progression of the condition from a localised condition to a widespread one involving the whole body in patients with fibromyalgia.

Overuse syndromes represent the low-grade spectrum of these conditions with expression determined by an increase in muscle activity. The increase in activity of the muscles results in increased energy use which is reduced due to the underlying problems. The muscles expressing most of the problems are those with the highest energy demand, such as shoulders, neck, arms and wrists in typists, elbows in people playing tennis, facial and neck muscles in violinists and musicians people playing brass and woodwind instruments, etc.

Despite the prevalence of chronic neuromuscular pain, there is currently no effective therapy and accordingly, there is a need for new methods and agents for preventing and/or treating conditions characterised by chronic neuromuscular pain.

In the work leading up to the present invention the instant inventor has developed a composition which is effective in alleviating one or more of the symptoms associated with chronic neuromuscular pain.

### SUMMARY OF THE INVENTION

Throughout this specification, unless the context requires otherwise, the word “comprise”, and variations such as “comprises” and “comprising”, will be understood to imply the inclusion of a stated element or integer or step or group of elements or integers or steps but not the exclusion of any other elements or integer or step or group of elements or integers or steps.

In one aspect of the invention there is provided a composition comprising an amino acid for use in the prophylaxis and/or treatment of one or more symptoms of chronic neuromuscular pain.

Another aspect of the present invention provides a composition comprising an amino acid together with an oligosaccharide and/or monosaccharide for use in the prophylaxis and/or treatment of one or more symptoms of chronic neuromuscular pain.

In a related aspect, the present invention provides a composition comprising:
i) asparagine;
ii) a branched chain amino acid;
iii) an amino acid selected from the group consisting of alanine, aspartate and glutamine; and
iv) an amino acid selected from the group consisting of arginine, lysine and ornithine;
   optionally together with at least one of:
v) a glucose-containing oligosaccharide; and
vi) a monosaccharide
   for use in the prophylaxis and/or treatment of one of more symptoms of chronic neuromuscular pain.

In another related aspect, the present invention provides a method for the prophylaxis and/or treatment of one or more symptoms of chronic neuromuscular pain in a subject, said method comprising administering to said subject an effective amount of a composition comprising an amino acid.

In a further related aspect, the present invention provides a method for the prophylaxis and/or treatment of one or more symptoms of chronic neuromuscular pain in a subject, said method comprising administering to said subject an effective amount of a composition comprising an amino acid together with an oligosaccharide and/or monosaccharide.

In yet a further related aspect, the present invention provides a method for the prophylaxis and/or treatment of one or more symptoms of chronic neuromuscular pain in a subject, said method comprising administering to said subject an effective amount of a composition comprising:
i) asparagine;
ii) a branched chain amino acid;
iii) an amino acid selected from the group consisting of alanine, aspartate and glutamine; and
iv) an amino acid selected from the group consisting of arginine, lysine and ornithine;
   optionally together with at least one of:
v) a glucose-containing oligosaccharide; and
vi) a monosaccharide
   for a time and under conditions sufficient to prevent or treat one or more symptoms of chronic neuromuscular pain.

Still even yet a further aspect of the present invention provides the use of a composition comprising;
i) asparagine;
ii) a branched chain amino acid;
iii) an amino acid selected from the group consisting of alanine, aspartate and glutamine; and
iv) an amino acid selected from the group consisting of arginine, lysine and ornithine;
   optionally together with at least one of:
v) a glucose-containing oligosaccharide; and
vi) a monosaccharide
   for the prophylaxis and/or treatment of one or more symptoms of chronic neuromuscular pain.

Yet even a further aspect of the present invention contemplates the use of:
i) asparagine;
ii) a branched chain amino acid;
iii) an amino acid selected from the group consisting of alanine, aspartate and glutamine; and
iv) an amino acid selected from the group consisting of arginine, lysine and ornithine;
   optionally together with at least one of:
v) a glucose-containing oligosaccharide; and
vi) a monosaccharide
   in the manufacture of a medicament for prophylaxis and/or treatment of one of more symptoms of chronic neuromuscular pain.

### DETAILED DESCRIPTION OF THE INVENTION

The present invention is predicated, in part, on the finding by the inventor that chronic neuromuscular pain is associated with amino-acidaemina in tissues such as muscle tissues. Such a fall in amino acid availability leads to an increased dependence upon glucose and an inhibition of oxidative phosphorylation.

Accordingly, one aspect of the invention provides a composition comprising an amino acid for use in the prophylaxis and/or treatment of one of more symptoms of chronic neuromuscular pain.

Another aspect of the present invention provides a composition comprising an amino acid together with an oligosaccharide and/or monosaccharide for use in the prophylaxis and/or treatment of one of more symptoms of chronic neuromuscular pain.

The phrase “chronic neuromuscular pain” is used herein in its broadest sense to include a range of conditions or syndromes such as, for example, fibromylagia, myofascial pain, repetitive strain injury or overuse syndromes, and cytokine-mediated cancer and chemotherapy associated pain.

The phrase “symptoms of chronic neuromuscular pain” will be well known to those skilled in the art and reference herein is a reference to a wide range of symptoms including, for example, neuropathic and nociceptive pain, muscle soreness, weakness, tiredness, numbness, tenderness, stiffness, tingling and the like.

In yet another aspect, the present invention provides a composition comprising:
i) asparagine;
ii) a branched chain amino acid;
iii) an amino acid selected from the group consisting of alanine, aspartate and glutamine; and
iv) an amino acid selected from the group consisting of arginine, lysine and ornithine; optionally together with at least one of:
v) a glucose-containing oligosaccharide; and
vi) a monosaccharide
   for use in the prophylaxis and/or treatment of one of more symptoms of chronic neuromuscular pain.

Reference herein to a particular “amino acid” includes reference to functional derivatives, homologues, chemical analogues and mimetics thereof.

Reference herein to a “branched chain amino acid” includes naturally occurring and non-naturally occurring functional branched chain amino acids. Particularly preferred naturally occurring branched chain amino acids are leucine, valine and isoleucine. A most particularly preferred branched chain amino acid is leucine. Without limitation to any particular mode or theory of operation, branched chain amino acids are believed to modulate protein degradation and are an important energy source.

Reference herein to an “oligosaccharide” includes reference to any hydrolysable polymer of one or more type of monosaccharides, said oligosaccharide containing from about 2 to 100 or more molecules of monosaccharide. Reference to an oligosaccharide includes reference to a disaccharide and a complex oligosaccharide.

Preferred glucose-containing oligosaccharides are dextrose and maltodextrins, particularly a corn maltodextrin.

Suitable monosaccharides will be well known to persons skilled in the art. Preferred monosaccharides are fructose and glucose.

Again, although not limiting the present invention, it is proposed herein that chronic neuromuscular pain is associated with amino acidaemia which results in a fall in intracellular protein levels in various tissues including muscle tissue. This in turn leads to an increased dependence on glucose and an inhibition of oxidative phosphorylation. The present composition alleviates chronic neuromusular pain by addressing this metabolic imbalance. Alanine, aspartate or glutamine modulates transfer of amino acids between muscle and liver. Arginine, lysine or ornithine modulate urea cycle activity; and asparagine modulates oxidative phosphorylation. The administration of a composition comprising these amino acids provides the means to correct the metabolic imbalance or amino acidaemia which is believed to be associated with chronic neuromuscular pain. Furthermore, a carbohydrate source in the form of a glucose-containing oligosaccharide together with a monosaccharide such as fructose or glucose assists to maximise the effect of the composition. Fructose is conveniently included to overcome the possible inhibition of glycolysis and a failure to use glucose efficiently.

In a further aspect, the present invention provides a method for the prophylaxis and/or treatment of one or more symptoms of chronic neuromuscular pain in a subject, said method comprising administering to said subject an effective amount of a composition comprising an amino acid.

In another aspect, the present invention provides a method for the prophylaxis and/or treatment of one or more symptoms of chronic neuromuscular pain in a subject, said method comprising administering to said subject an effective amount of a composition comprising an amino acid together with an oligosaccharide and/or monosaccharide.

In a further related aspect, the present invention provides a method for the prophylaxis and/or treatment of one or more symptoms of chronic neuromuscular pain in a subject, said method comprising administering to said subject an effective amount of a composition comprising:
i) asparagine;
ii) a branched chain amino acid;
iii) an amino acid selected from the group consisting of alanine, aspartate and glutamine; and
iv) an amino acid selected from the group consisting of arginine, lysine and ornithine;
   optionally together with at least one of:
v) a glucose-containing oligosaccharide; and
vi) a monosaccharide
   for a time and under conditions sufficient to prevent or treat one or more symptoms of chronic neuromuscular pain.

Components of the instant compositions may be derived from any convenient source provided they are effective in combination in preventing or treating one or more symptoms of chronic neuromuscular pain. For example, the components may be in purified or isolated form. By “isolated form” is meant that the component amino acid or carbohydrate has undergone at least one step of purification from a biological source. Preferably, the component is at least about 20%, more preferably at least about 40%, still more preferably about 65%, even still more preferably about 80-90% or greater pure as determined by activity or other convenient means.

The compositions may be orally administered, for example, with an inert diluent or with an assimilable edible carrier, or it may be enclosed in hard or soft shell gelatin capsule, or it may be compressed into tablets, or it may be in powdered form or incorporated directly with the food of the diet. For oral therapeutic and/or prophylactic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.01 μg and about 2000 mg of active compound. Alternative amounts include between about 1.0 μg and about 1500 ng, between about 1 μg and about 1000 mg and between about 10 μg and about 500 mg.

The present invention expressly contemplates oral administration of a convenient composition as herein described.

The tablets, troches, pills, capsules and the like may also contain the components as listed hereafter: A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

Parenteral compositions are also contemplated. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of pain in a wide range of subjects.

The principal active ingredient or ingredients are compounded for convenient and effective administration in effective amounts with a suitable pharmaceutically acceptable carrier in dosage unit form. A unit dosage form can, for example, contain the principal active compounds in amounts ranging from 0.01 μg to about 70 g/100 grams. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients. Alternatively, amounts administered may be represented in terms of amounts/kg body weight. In this case, amounts range from about 0.001 μg to about 1000 mg/kg body weight may be administered. Preferably amounts rang from 500 ug to 500 mg/kg body weight or about 0.1 μg to about or above 10 μg to about 250 mg/kg body weight are contemplated by the present invention.

In yet a further aspect of the present invention provides the use of a composition comprising;
i) asparagine;
ii) a branched chain amino acid;
iii) an amino acid selected from the group consisting of alanine, aspartate and glutamine; and
iv) an amino acid selected from the group consisting of arginine, lysine and ornithine;
   optionally together with at least one of:
v) a glucose-containing oligosaccharide; and
vi) a monosaccharide
   for the prophylaxis and/or treatment of one or more symptoms of chronic neuromuscular pain.

Yet even a further aspect of the present invention contemplates the use of:
i) asparagine;
ii) a branched chain amino acid;
iii) an amino acid selected from the group consisting of alanine, aspartate and glutamine; and
iv) an amino acid selected from the group consisting of arginine, lysine and ornithine;
   optionally together with at least one of:
v) a glucose-containing oligosaccharide; and
vi) a monosaccharide
   in the manufacture of a medicament for prophylaxis and/or treatment of one of more symptoms of chronic neuromuscular pain.

The present invention is now further described with reference to the following non-limiting examples.

### EXAMPLE 1

The following composition was tested in subjects:

| Compound | mg per 10 Grams | |
|---|---|---|
| Corn maltodextrins | 5504.6 | mg |
| Dextrose monohydrate | 1376.1 | mg |
| Fructose | 917.4 | mg |
| L-Alanine | 540.1 | mg |
| L-Leucine | 226.1 | mg |
| L-Isoleucine | 226.1 | mg |
| L-Valine | 257.2 | mg |
| L-Glycine | 191.7 | mg |
| L-Asparagine | 93.4 | mg |
| L-Threonine | 93.4 | mg |
| L-Serine | 67.1 | mg |
| L-Proline | 52.4 | mg |
| L-Glutamic acid | 41.0 | mg |
| L-Aspartic acid | 37.1 | mg |
| L-Lysine | 14.9 | mg |
| L-Arginine | 12.0 | mg |
| L-Tyrosine | 11.2 | mg |
| L-Histidine | 11.2 | mg |
| L-Methionine | 11.2 | mg |
| L-Phenylalanine | 11.2 | mg |
| Taurine | 11.2 | mg |
| L-Cystine | 11.2 | mg |
| L-Ornithine | 10.0 | mg |
| Calcium succinate | 9.8 | mg |
| L-Tryptophan | 7.0 | mg |
| Magnesium citrate | 7.0 | mg |
| Ascorbic acid | 239.1 | mg |
| Nicotinamide | 2.4 | mg |
| d-alpha Tocopheryl acetate | 1.8 | mg |
| Ferrous fumarate | 1.8 | mg |
| α-Lipoic acid | 0.6 | mg |
| Calcium Pantothenate | 0.6 | mg |
| Riboflavine | 0.6 | mg |
| Thiamine | 0.6 | mg |
| Betacarotene | 0.4 | mg |
| Biotin | 0.2 | mg |
| Cholecalciferol | 0.2 | mg |
| Cyanocobalamin | 0.2 | mg |

### EXAMPLE 2

A 300 mg capsule of the supplement of Example 1 was given to a 35 year-old female patient with myofascial pain syndrome. One week later the patient reported that the pain and muscle tenderness had diminished significantly and were now virtually all gone. The patient also reported that there were significant improvements in her cognitive abilities and mood. The patient continues to take the mixture as it prevents the recurrence of most of her symptoms.

### EXAMPLE 3

A 300 mg capsule of the supplement of Example 1 was given to a 53 year old female who was undergoing chemotherapy following breast cancer. The patient had significant fatigue, cognitive disturbance and musculoskeletal pain which are standard symptoms noted following chemotherapy. Within several days her pain, cognitive abilities and mood had all improved to the point that she was able to go back to work. This supplement was given during 3 episodes of chemotherapy which formed part of her total chemotherapy regimen and resulted in significant reductions in her musculoskeletal, cognitive disturbance and mood changes.

### EXAMPLE 4

A 36 year old male who had arm and shoulder pain which was exacerbated by his job which included holding his arms out consistently whilst working. The pain he experienced disappeared after taking a 300 mg capsule per day of the supplement of Example 1 for 4 to 5 days.

### EXAMPLE 5

The following composition is also tested in subjects:

| Compound | mg per 10 Grams | |
|---|---|---|
| Corn maltodextrins | 5504.6 | mg |
| Dextrose monohydrate | 1376.1 | mg |
| Fructose | 917.4 | mg |
| L-Alanine | 540.1 | mg |
| L-Leucine | 226.1 | mg |
| L-Isoleucine | 226.1 | mg |
| L-Valine | 257.2 | mg |
| L-Glycine | 191.7 | mg |
| L-Asparagine | 93.4 | mg |
| L-Threonine | 93.4 | mg |
| L-Serine | 67.1 | mg |
| L-Proline | 52.4 | mg |
| L-Glutamic acid | 41.0 | mg |
| L-Aspartic acid | 37.1 | mg |
| L-Lysine | 14.9 | mg |
| L-Tyrosine | 11.2 | mg |
| L-Histidine | 11.2 | mg |
| L-Phenylalanine | 11.2 | mg |
| Taurine | 11.2 | mg |
| L-Cystine | 11.2 | mg |
| L-Ornithine | 10.0 | mg |
| Calcium succinate | 9.8 | mg |
| L-Tryptophan | 7.0 | mg |
| Magnesium citrate | 7.0 | mg |
| Ascorbic acid | 239.1 | mg |
| Nicotinamide | 2.4 | mg |
| d-alpha Tocopheryl acetate | 1.8 | mg |
| Ferrous fumarate | 1.8 | mg |
| α-Lipoic acid | 0.6 | mg |
| Calcium Pantothenate | 0.6 | mg |
| Riboflavine | 0.6 | mg |
| Thiamine | 0.6 | mg |
| Betacarotene | 0.4 | mg |
| Biotin | 0.2 | mg |
| Cholecalciferol | 0.2 | mg |
| Cyanocobalamin | 0.2 | mg |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

De Girolamo, G., Clin J Pain, 7:S1-S7, 1991.

Waylonis, G W., Heck, W, Am J Phys Med Rehab 71:343-348, 1992.

What is claimed is:

1. A composition comprising an amino acid for use in the prophylaxis and/or treatment of one or more symptoms of chronic neuromuscular pain.

2. A composition comprising an amino acid together with an oligosaccharide and/or monosaccharide for use in the prophylaxis and/or treatment of one or more symptoms of chronic neuromuscular pain.

3. A composition according to claim 1 comprising:
i) asparagine;
ii) a branched chain amino acid;
iii) an amino acid selected from the group consisting of alanine, aspartate and glutamine; and
iv) an amino acid selected from the group consisting of arginine, lysine and ornithine;
optionally together with at least one of:
v) a glucose-containing oligosaccharide; and
vi) a monosaccharide
for use in the prophylaxis and/or treatment of one of more symptoms of chronic neuromuscular pain.

4. A composition according to claim 3 wherein said branched chain amino acid is leucine, valine and/or isoleucine.

5. A composition according to claim 3 wherein said glucose-containing oligosaccharide is dextrose and/or maltodextrin.

6. A composition according to claim 3 wherein said monosaccharide is fructose and/or glucose.

7. A composition according to claim 1 when used in the prophylaxis and/or treatment of one of more symptoms of chronic neuromuscular pain.

8. A method for the prophylaxis and/or treatment of one or more symptoms of chronic neuromuscular pain in a subject, said method comprising administering to said subject an effective amount of a composition comprising an amino acid.

9. A method for the prophylaxis and/or treatment of one or more symptoms of chronic neuromuscular pain in a subject, said method comprising administering to said subject an effective amount of a composition comprising an amino acid together with an oligosaccharide and/or monosaccharide.

10. A method according to claim 8 wherein said composition comprises:
i) asparagine;
ii) a branched chain amino acid;
iii) an amino acid selected from the group consisting of alanine, aspartate and glutamine; and
iv) an amino acid selected from the group consisting of arginine, lysine and ornithine;
optionally together with at least one of:
v) a glucose-containing oligosaccharide; and
vi) a monosaccharide
for a time and under conditions sufficient to treat or prevent one of more symptoms of chronic neuromuscular pain.

11. A method according to 10 wherein said branched chain amino acid is leucine, valine and/or isoleucine.

12. A method according to claim 10 wherein said glucose-containing oligosaccharide is dextrose and/or maltodextrin.

13. A method according to claim 10 wherein said monosaccharide is fructose and/or glucose.

14. A method according to claim 13 wherein said monosaccharide is fructose.

15. A method according to claim 10 wherein said subject is a human subject.

16. Use of:
i) asparagine;
ii) a branched chain amino acid,
iii) an amino acid selected from the group consisting of alanine, aspartate and glutamine; and
iv) an amino acid selected from the group consisting of arginine, lysine and ornithine;
optionally together with at least one of:
v) a glucose-containing oligosaccharide; and
vi) a monosaccharide
in the manufacture of a medicament for the prophylaxis and/or treatment of one or more symptoms of chronic neuromuscular pain.

17. A use according to claim 16 wherein said branched chain amino acid is leucine, valine and/or isoleucine.

18. A use according to claim 16 wherein said glucose-containing oligosaccharide is dextrose and/or maltodextrin.

19. A use according to claim 16 wherein said monosaccharide is fructose and/or glucose.

20. A use according to claim 19 wherein said monosaccharide is fructose.
